(19) United States
(12) Patent Application Publication
(10) Pub. No.: US 2007/0027360 A1
(43) Pub. Date: Feb. 1, 2007

(54) Endoscope

(75) Inventor: Tae Mitsuya, Sagamihara-shi (JP)

Correspondence Address: SCULLY SCOTT MURPHY & PRESSER, PC, 400 GARDEN CITY PLAZA, SUITE 300, GARDEN CITY, NY, 11530, US

(73) Assignee: Olympus Corporation, Tokyo (JP)

(21) Appl. No.: 11/506,138

(22) Filed: Aug. 17, 2006

Related U.S. Application Data
(63) Continuation of application No. PCT/JP05/02634, filed on Feb. 18, 2005.

(30) Foreign Application Priority Data
Feb. 20, 2004 (JP) 2004-045189

(51) Int. Cl. A61B 1/00; A61B 1/12

(52) U.S. Cl. 600/129; 600/141; 600/130; 600/157

(57) ABSTRACT

An endoscope includes in an insertion portion thereof a hard distal end portion and a bending portion arranged consecutively with the distal end portion; with the center axes of the distal end portion and the bending portion kept eccentric with each other, the distal end portion and the bending portion are coupled. A distal-end constituent member includes a distal-end bending-portion joint piece arrangement stage, a transition portion, a distal-end formation portion, and the like. The distal-end formation portion defines the contour of the distal-end constituent member, and the center position is OA. The transition portion is the reference plane for the bending portion, and the center position is OB. A distal-end bending-portion joint piece incorporated in the bending portion is arranged on the distal-end bending-portion joint piece arrangement stage. The center positions of the distal-end formation portion and the transition portion are eccentric with each other.

### CROSS REFERENCE TO RELATED APPLICATION

This application is a continuation application of PCT/JP2005/002634 filed on Feb. 18, 2005 and claims benefit of Japanese Application No. 2004-045189 filed in Japan on Feb. 20, 2004, the entire contents of which are incorporated herein by this reference.

### BACKGROUND OF THE INVENTION

1. Field of the Invention

The present invention relates to an endoscope in which a hard distal end portion incorporated in an insertion portion and a bending portion are consecutively arranged.

2. Description of the Prior Art

In recent years, endoscope systems have been commonly utilized that make it possible to insert an insertion portion into a body cavity or a pipe and implement observation, while displaying a subject image on a screen of a display apparatus. In general, endoscopes utilized in the foregoing endoscope systems include a distal end portion and a bending portion in the distal end of an elongated insertion portion. By providing the bending portion in the insertion portion of the endoscope and bending the bending portion, the distal end portion is oriented to a desired direction.

The distal end portion of the endoscope is formed of a hard material. In the distal end portion, an illumination optical system for illuminating an examination site and an image-capturing optical system for obtaining the observation image of the examination site illuminated by the illumination optical system are arranged. In addition, in an endoscope for the purpose of implementing not only observation but also treatment, a treatment-device channel for introducing a treatment device into the body cavity is provided, in addition to the illumination optical system and the observation optical system.

From the distal end portion of the endoscope, a bundle of light-guide fibers incorporated in the illumination optical system, part of an image-capturing unit incorporated in the observation optical system, and a signal cable that extends from the image-capturing unit, and, as may be necessary, a channel port metal incorporated in the treatment-device channel, a channel tube one end of which is fixed to the channel port metal, and the like extend outward.

The bending portion of the endoscope is configured of a plurality of bending-portion joint pieces, which are arranged pivotably and consecutively with one another, in such a way as to be able to freely bend up and down or up and down/right and left. By integrally fixing to the proximal portion of the distal end portion a distal-end bending-portion joint piece incorporated in the foremost end of the bending portion, the bending portion and the distal end portion are consecutively arranged.

An endoscope having an insertion portion configured in such a way that, as described above, the bending portion and the distal end portion are consecutively arranged employs a configuration in which the distal-end bending-portion joint piece arranged at the proximal side of the distal end portion and the distal end portion are arranged approximately coaxially with each other. Accordingly, if the bending portion is viewed from the distal endface of the distal end portion, an outline variation portion is seen in which the circumference of the bending portion is approximately evenly larger than that of the distal end portion. Although the dimensions of outer diameters in the bending portion and the distal end portion are different in the center and the diameter dimension of the circumscribing circle of the constituent element, the reason is represented by making the circumscribing circles so as to satisfy the both.

### SUMMARY OF THE INVENTION

The present invention provides an endoscope in which a hard distal end portion and a bending portion are arranged consecutively with each other at the distal side of the insertion portion. When the center axes of the distal end portion and the bending portion are coupled, the center axes of the distal end portion and the bending portion are eccentric with each other.

### BRIEF DESCRIPTION OF THE DRAWINGS

FIG. 1 is a diagram for explaining an endoscope system including an endoscope according to the present invention;

FIG. 2 is a view for explaining the configuration of the distal endface of an insertion portion;

FIG. 3 is a front view illustrating a distal-end constituent member, viewed from the distal endface;

FIG. 4 is a side view illustrating a distal-end constituent member;

FIG. 5 is a cross-sectional view of the distal-end constituent member, taken along the line V-V in FIG. 4;

FIG. 6 is a longitudinal cross-sectional view for explaining the principal parts of an endoscope;

FIG. 7 is a cross-sectional view of the principal parts of an endoscope, taken along the line VII-VII in FIG. 6;

FIG. 8 is a plan view for explaining an air/water feed nozzle;

FIG. 9 is a cross-sectional view of the air/water feed nozzle, taken along the line IX-IX in FIG. 8;

FIG. 10 is a front view illustrating a distal-end constituent member, viewed from the proximal endface; and

FIG. 11 is a cross-sectional view of the principal parts of an endoscope, taken along the line XI-XI in FIG. 6.

### DETAILED DESCRIPTION OF THE PREFERRED EMBODIMENTS

An embodiment of the present invention will be explained below, with reference to the drawings.

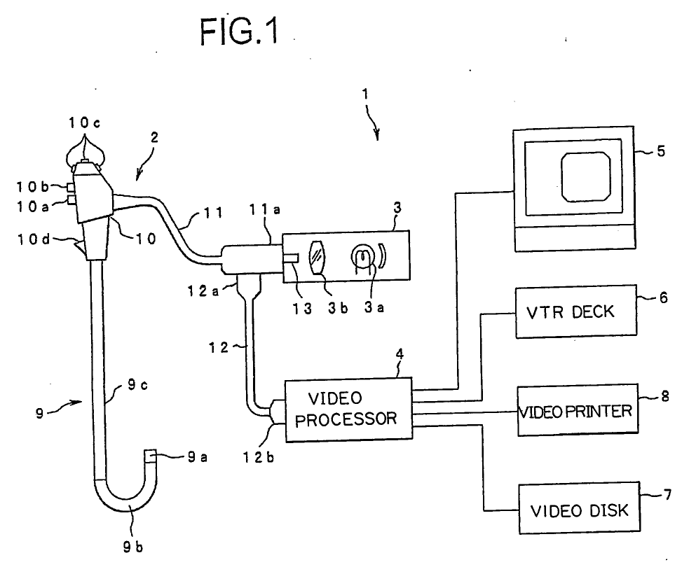

As illustrated in FIG. 1, an endoscope system 1 is configured of an endoscope 2, a light source device 3, a video processor 4, a display monitor 5, a VTR deck 6, a video disk 7, and a video printer 8.

The endoscope 2 incorporates an image-capturing unit, described later, having an image-capturing element (not shown), for example, for obtaining observation images of an examination site. The light source device 3 supplies the endoscope 2 with illumination light. The video processor 4 implements the control of the endoscope 2, the signal processing of image signals obtained through the endoscope 2, and the like. The display monitor 5 displays observation images, based on video signals outputted from the video processor 4. The VTR deck 6 and the video disk 7 record observation images. The video printer 8 prints out observation images.

The endoscope 2 is configured mainly of an elongated insertion portion 9, a operation unit 10 situated in the proximal portion of the insertion portion 9, and a universal cord 11 extending from a side of the operation unit 10. The insertion portion 9 is formed, by consecutively arranging a distal end portion 9a, a bending portion 9b, and a flexible tube portion 9c in that order from the distal end of the insertion portion 9.

The distal end portion 9a is formed of a hard material; for example, an illumination optical system and an observation optical system, described later, are arranged therein. The bending portion 9b is configured of a plurality of bending-portion joint pieces arranged consecutively in such a way as to be able to bend, e.g., up and down. The flexible tube portion 11 has flexibility.

In the operation unit 10, for example, an unillustrated bending knob for making the bending portion 9b bend, an air/liquid feed button 10a for controlling an air/liquid feed function, a suction button 10b for controlling a suction function, a switch 10c for remotely implementing a video recording function of the video processor 4 and light-amount adjustment for the light source device 3, and the like are provided. In addition, a treatment-device insertion port 10d is provided through which a treatment device such as a piece of hand-held forceps is introduced into the body cavity.

A light guide for transmitting illumination light, a signal line for transmitting electric signals, and the like are inserted through the universal cord 11. At one end of the universal cord 11, a light-source connector 11 a is provided. The light-source connector 11 a and the light source device 3 are detachably and attachably connected with each other. A connector 12a provided at one end of a signal cable 12 is detachably and attachably connected to the side portion of the light-source connector 11a. A connector 12b provided at the other end of the signal cable 12 is detachably and attachably connected to the video processor 4.

Inside the light source device 3, a lamp 3a is provided. Illumination light from the lamp 3a passes through a condenser lens 3b and then is converged onto the endface of a light-guide port metal 13 protruding from the light-source connector 11a. The illumination light converged onto the endface of the light-guide port metal 13 is introduced to the distal end portion 9a of the insertion portion 9, by way of the universal cord 11 and a bundle of light-guide fibers that is inserted through the endoscope 2, and irradiated from the illumination optical system onto an examination site.

The optical image of the examination site onto which the illumination light is irradiated passes through an objective lens provided in the distal end portion 9a and formed on an image-capturing element provided in the image-capturing unit. The image-capturing element converts the formed optical image into an electric signal and transmits the electric signal to the video processor 4, by way of the signal line. The video processor 4 converts the transmitted electric signal into a video signal and outputs the video signal to the display monitor 5. Accordingly, an endoscopic image captured by the image-capturing element is displayed on the display monitor 5.

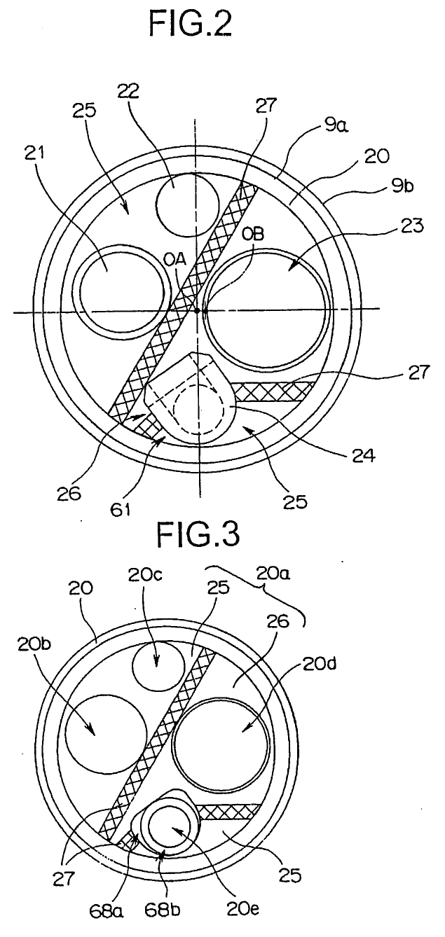

As illustrated in FIG. 2, an objective lens 21, an illumination lens 22, a treatment-device opening 23, and an air/water feed nozzle 24 are provided at respective predetermined positions on a distal endface 20a of a distal-end constituent member 20 that is incorporated in the distal end portion 9a of the insertion portion 9. The objective lens 21 is an observation window incorporated in the observation optical system. The illumination lens 22 is an illumination window incorporated in the illumination optical system. The treatment-device opening 23 communicates to the treatment-device insertion port 10d; the treatment device that is inserted from the treatment-device insertion port 10d passes through and goes out from the treatment-device opening 23. The emission outlet of the air/water feed nozzle 24 faces the objective lens 21. In addition, to the surface of the curved portion of the illumination lens 22, formed of an optical material, surface treatment is applied with which light is diffused due to refraction.

As illustrated in FIG. 3, in the distal-end constituent member 20, an observation-optical-system through-hole 20b, an illumination-optical-system through-hole 20c, a treatment-device channel through-hole 20d, and a nozzle-arrangement hole 20e are formed. In the observation-optical-system through-hole 20b, the observation optical system such as the objective lens 21 is arranged. In the illumination-optical-system through-hole 20c, the illumination optical system such as the illumination lens 22 is arranged. The opening of the treatment-device channel through-hole 20d is the treatment-device opening 23. In the nozzle-arrangement hole 20e, the air/water feed nozzle 24 is arranged. The endoscope 2 according to the present embodiment is a so-called direct-view type endoscope configured in such a way that observation is implemented ahead in a direction in which the insertion portion 9 is inserted; the optical axis of the observation optical system is approximately in parallel with the longitudinal axis of the insertion portion 9.

As illustrated in FIGS. 2 to 4 and 6, in consideration of observation performance and cleaning performance, the distal endface 20a of the distal-end constituent member 20 is formed, in a stepped contour, provided with a first plane 25 and a second plane 26 that are different from each other in level. Slopes 27, in each of FIGS. 2 and 3, indicated by the crosshatch are provided between the first plane 25 and the second plane 26.

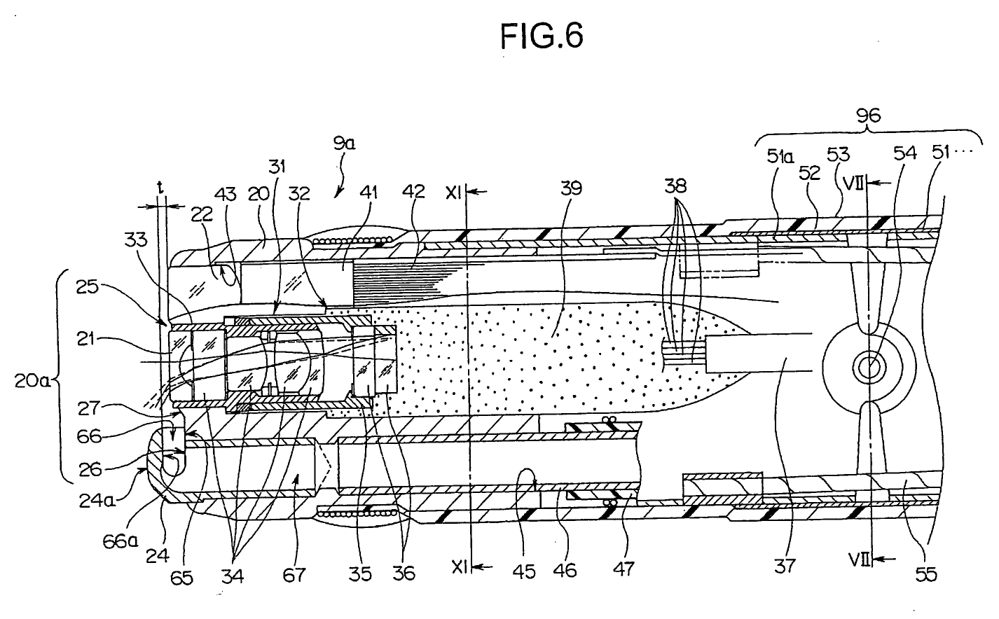

As illustrated in FIG. 6, in the proximal portion of the objective lens 21, an objective unit 31 incorporated in the observation optical system and an image-capturing unit 32 as an image-capturing device are provided. The objective unit 31 is configured of an objective lens frame 33, the objective lens 21 disposed in the objective lens frame 33, and optical lenses 34. In contrast, the image-capturing unit 32 is configured of an image-capturing frame 35, a plurality of optical lenses 36 disposed in the image-capturing frame 35, and an unillustrated image-capturing element, a circuit board on which electronic components are mounted, and the like that are disposed in the proximal portion of the optical lens 36.

Reference numeral 37 denotes a signal cable that extends from the image-capturing unit 32. Various kinds of signal wires 38 are inserted through the signal cable 37. Reference numeral 39 denotes a sealing resin that seals the image-capturing element, the circuit board, the signal wires 38, and the like.

In the proximal portion of the illumination lens 22, a rod lens 41 and the bundle of light-guide fibers 42 are disposed. The illumination lens 22 is fixed to the illumination-optical-system through-hole 20c, by means of an adhesive. Adhesion puddle 43 is provided at a predetermined position in the illumination-optical-system through-hole 20c to which the illumination lens 22 is fixed. By providing the adhesion puddle 43 in the illumination-optical-system through-hole 20c, it is possible to prevent the adhesive from flowing on the surface, of the curved portion, to which surface treatment is applied, when the illumination lens 22 is adhered to the through-hole 20c.

The air/water feed nozzle 24 is formed approximately in an L shape. An air/water feed through-hole 45 is formed in the proximal portion of the air/water feed nozzle 24. A water feed port metal 46 is disposed in the air/water feed through-hole 45; one end of a water feed tube 47 is coupled with the water feed port metal 46.

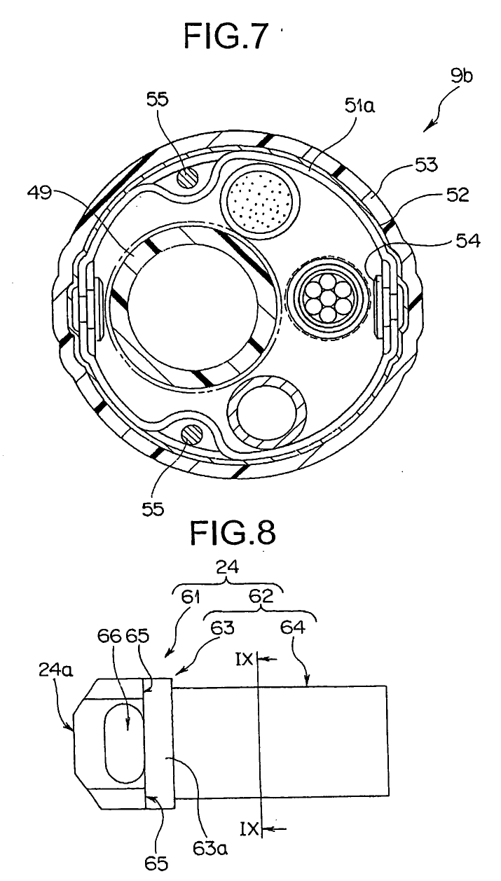

In addition, in the proximal portion of the treatment-device opening 23, a treatment-device-channel port metal (refer to reference numeral 48, in FIG. 1, described later) is provided. One end of a treatment-device channel tube (refer to reference numeral 49 in FIG. 7) is coupled with the treatment-device-channel port metal 48.

As illustrated in FIGS. 6 and 7, the bending portion 9b is configured mainly of a plurality of bending-portion joint pieces 51, a net tube 52 for covering the plurality of bending-portion joint pieces 51, and a coating tube 53 for covering the net tube 52.

The bending-portion joint pieces 51 are coupled with one another, by means of connecting pins 54, pivotably and consecutively. The net tube 52 is formed, e.g., by knitting wires made of a polymeric material or metal, in a circular-tube fashion. The coating tube 53 is a rubber material, or an elastomer such as a urethane resin, that has high extensibility.

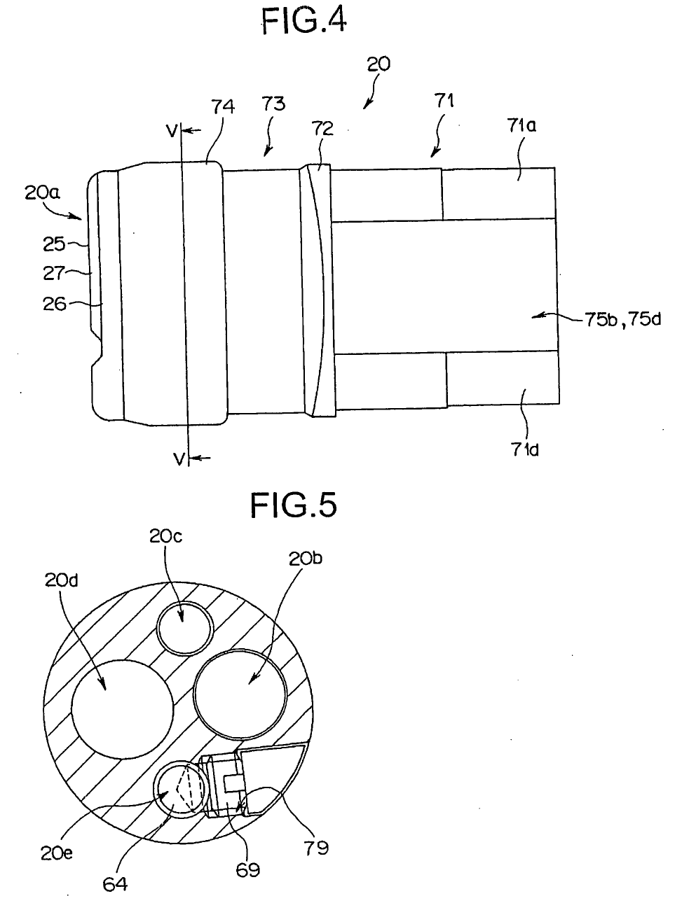

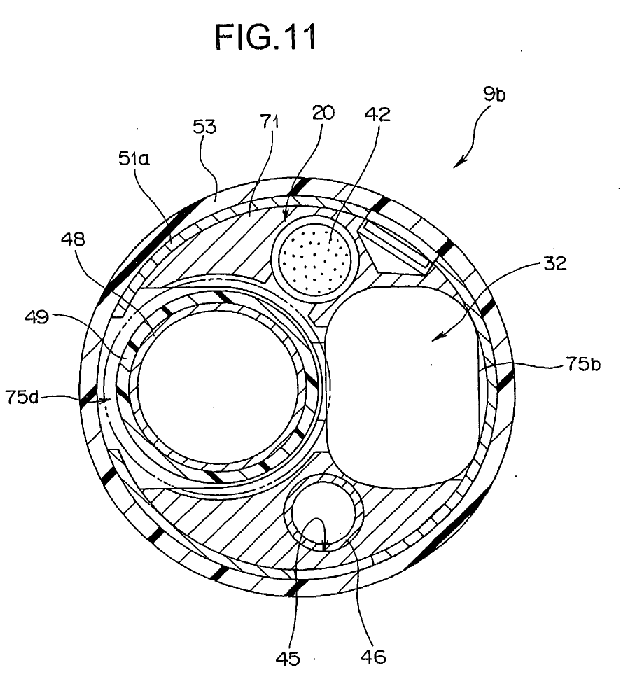

A distal-end bending-portion joint piece 51 a situated at the foremost end of the bending portion 9b is fixed being coupled with a distal-end bending-portion joint piece arrangement stage (refer to reference numeral 71, in FIGS. 4 and 11, described later) formed in the proximal portion of the distal-end constituent member 20. Additionally, an unillustrated bending-portion joint piece situated at the endmost position of the bending portion 9b is fixed being coupled with an unillustrated flexible-tube distal-end constituent member incorporated the flexible tube portion 9c.

A pair of bending operation wires (briefly referred to as a wire, hereinafter) 55 is integrally fixed, through soldering or the like, to a pair of predetermined positions on the inner surface of the distal-end bending-portion joint piece 51a. With regard to the arrangement position of the wire 55, the gravity-center position is taken into account so that, in implementing bending manipulation of the bending portion 9b, the bending-force amount becomes as light as possible. In the present embodiment, the wires 55 are situated above and below, in the drawing, the treatment-device channel tube 49. Accordingly, when, under the conditions that the treatment device is being inserted and not being inserted yet through the treatment-device channel tube 49, an operator manipulates the unillustrated bending knob, the bending portion is smoothly bent.

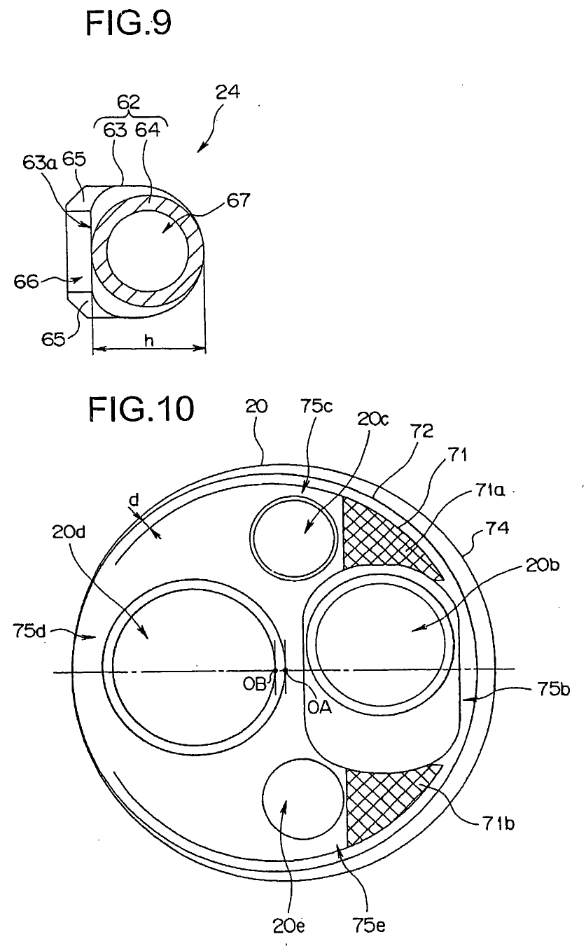

As illustrated in FIGS. 2, 6, 8, and 9, the L-shaped air/water feed nozzle 24 is configured of a nozzle portion 61 and a nozzle fixation portion 62. In the nozzle fixation portion 62, a thick-diameter fitting portion 63 and a cylindrical engagement portion 64 are provided. The fitting portion 63 and the cylindrical engagement portion 64 are equal to each other in “h” dimension. The fitting portion 63 is provided at the side, of the nozzle fixation portion 62, that is adjacent to the nozzle portion 61. The fitting portion 63 has an approximately D-shaped cross section and a flat portion 63a that plays the roles of rotation prevention portion and a positioning portion. The cylindrical engagement portion 64 is smaller in diameter than the fitting portion 63 and has a circular cross section. In addition, reference numeral 67 denotes a fluid hole communicating with a groove portion 66 described later.

The nozzle portion 61 is formed so as to protrude by a predetermined distance from the flat portion 63a. In addition, in the nozzle portion 61, an abutting surface 65 is provided that abuts on the second plane 26 of the distal-end constituent member 20. Moreover, in the nozzle portion 61, the groove portion 66 is formed that is formed by cutting off the middle part of the abutting surface 65 incorporated in the emission outlet.

As illustrated in FIG. 6, the level difference dimensions between the first plane 25 and the second plane 26 is formed in such a way that a distal endface 24a of the air/water feed nozzle 24 is out of the viewing angle of the observation optical system. In other words, the first plane 25 protrudes from the second plane 26 in the axis direction. In consequence, it is prevented that, under the condition that the fitting portion 63 and the cylindrical engagement portion 64 of the air/water feed nozzle 24 are disposed in the nozzle-arrangement hole 20e and the abutting surface 65 abuts on the second plane 26, the viewing angle of the observation optical system contains the distal endface 24a of the air/water feed nozzle 24 and the like.

In addition, the depth, of the groove portion 66, from the abutting surface 65 is set in such a way that, under the condition that, as described above, the air/water feed nozzle 24 is disposed in the nozzle-arrangement hole 20e and the abutting surface 65 abuts on the second plane 26, the groove-portion bottom side 66a is situated shifted by a predetermined dimension t from the surface of the objective lens 21 toward the distal end.

Accordingly, by, with the air/water feed nozzle 24 disposed in the distal-end constituent member 20, situating the groove-portion bottom side 66a incorporated in the emission outlet always shifted by the predetermined distance t from the surface of the objective lens 21 toward the distal end, provision is made for a fluid emitted from the emission outlet to evenly cover the surface of the objective lens 21.

As illustrated in FIG. 3, in the nozzle-arrangement hole 20e formed in the distal-end constituent member 20, a D-shaped hole 68a having a plane portion on which the flat portion 63a in the fitting portion 63 of the air/water feed nozzle 24 is arranged and a circular hole 68b in which the cylindrical engagement portion 64 is engaged and arranged are provided. The plane portion provided in the D-shaped hole 68a is formed at a position based on which, when the fitting portion 63 of the air/water feed nozzle 24 is disposed in the D-shaped hole 68a in the nozzle-arrangement hole 20e, the emission outlet faces the objective lens 21.

Accordingly, by disposing the nozzle fixation portion 62 of the air/water feed nozzle 24 in the nozzle-arrangement hole 20e, the emission outlet provided in the air/water feed nozzle 24 faces the objective lens 21 and is prevented from rotating.

After the air/water feed nozzle 24 is disposed in the nozzle-arrangement hole 20e, the air/water feed nozzle 24 is integrally fixed to the distal-end constituent member 20, by means of a fixation screw 69 as a fixation member, as illustrated in FIG. 5. The fixation screw 69 is screwed and disposed into a fixation hole 79 formed in the distal-end constituent member 20. Through screwing the fixation screw 69, the distal endface of the fixation screw 69 presses the peripheral face of the cylindrical engagement portion 64, whereby a predetermined fixation state is produced.

Next, the configuration of the distal-end constituent member 20 will be explained with reference to FIGS. 3, 4, 5, and 10.

The distal-end constituent member 20 illustrated in FIG. 4 is a cylindrical member. As illustrated in FIGS. 3, 5, and 10, in the distal-end constituent member 20, the observation-optical-system through-hole 20b, the illumination-optical-system through-hole 20c, the treatment-device channel through-hole 20d, and the nozzle-arrangement hole 20e, described above, are formed.

As illustrated in FIGS. 4 and 10, the distal-end constituent member 20 is configured mainly of the distal-end bending-portion joint piece arrangement stage 71, a transition portion 72, a coating-tube arrangement recess 73, and a distal-end formation portion 74 in that order from the proximal portion. The distal-end formation portion 74 defines the contour of the distal-end constituent member 20. The center position of the distal-end formation portion 74 is indicated by reference character OA. In the coating-tube arrangement recess 73, the distal end portion of the coating tube 53 is arranged. The coating-tube arrangement recess 73 is formed so as to cover the overall circumference. The transition portion 72 is the reference plane for the bending portion 9b; the center position of the transition portion 72 is indicated by reference character OB. The distal-end bending-portion joint piece 51a is arranged on the distal-end bending-portion joint piece arrangement stage 71. A level difference of a predetermined dimension d is formed between the peripheral face of the distal-end bending-portion joint piece arrangement stage 71 and the peripheral face of the transition portion 72. In other words, the radius of the distal-end bending-portion joint piece arrangement stage 71 is smaller by the dimension d than that of the transition portion 72.

That is to say, in the distal-end constituent member 20, the center position OA for defining the contour of the distal-end formation portion 74 and the center position OB for defining the peripheral face of the distal-end bending-portion joint piece arrangement stage 71 on which the distal-end bending-portion joint piece 51a is arranged are made eccentric with each other (also described “offset from each other”). Accordingly, when the distal-end bending-portion joint piece 51a is arranged on the distal-end bending-portion joint piece arrangement stage 71 of the distal-end constituent member 20 to configure the insertion portion 9, as shown in FIG. 2, the center position OB of the bending portion 9b is eccentric with the center position OA of the distal end portion 9a; therefore, when being disposed in the circumference of the distal end portion 9a, the bending portion 9b is disposed in such a way that the circumference of the bending portion 9b is eccentric.

The spacing between the inner surface of the distal-end bending-portion joint piece 51a and the image-capturing unit 32 and the spacing between the inner surface of the distal-end bending-portion joint piece 51a and the treatment-device channel tube will be compared between the case where, with the configuration in which the center positions OA and OB are offset with each other, the distal-end bending-portion joint piece 51a is arranged on the distal-end bending-portion joint piece arrangement stage 71 and the case where, with the configuration in which the center positions OA and OB are coaxial with each other, the distal-end bending-portion joint piece 51a is arranged on the distal-end bending-portion joint piece arrangement stage 71. The comparison results in the fact that the positional relationship in the case of the offset configuration is imbalanced toward one side, compared to the positional relationship in the case of the coaxial configuration.

In the present embodiment, the endoscope is configured in such a way that the image-capturing unit is arranged within a region that is different from the offset-side region in which the middle-side, of the bending portion, that is made offset with respect to the center axis of the distal end portion is situated. Accordingly, an imbalance is produced in which the spacing between the inner surface of the distal-end bending-portion joint piece 51a and the image-capturing unit 32 is narrowed and the spacing between the inner surface of the distal-end bending-portion joint piece 51a and the treatment-device channel tube is widened. Thus, in the configuration in which the center positions OA and OB are offset from each other, the distal-end bending-portion joint piece 51a is formed in such a way that the diameter thereof is small, in order to solve the imbalance. Accordingly, the spacing between the inner surface of the distal-end bending-portion joint piece 51a and the treatment-device channel tube is narrowed, without changing the spacing between the inner surface of the distal-end bending-portion joint piece 51a and the image-capturing unit 32, whereby the state is solved in which the distal-end bending-portion joint piece 51a is offset with respect to the distal-end formation portion 74.

In addition, in order to reduce the diameter of the bending portion 9b, in the distal-end bending-portion joint piece arrangement stage 71, notched portions 75b, 75c, 75d, and 75e are formed that are for removing the peripheral walls, i.e., the so-called side walls of the observation-optical-system through-hole 20b, the illumination-optical-system through-hole 20c, the treatment-device channel through-hole 20d, and the nozzle-arrangement hole 20e. By removing the peripheral walls to form the notched portions 75b, 75c, 75d, and 75e, the eccentricity, of the center position OB, with respect to the center position OA is made enlarged, whereby the diameter of the distal-end bending-portion joint piece 51a can further be reduced.

Specifically, as illustrated in FIG. 11, in the observation-optical-system through-hole 20b, the illumination-optical-system through-hole 20c, the treatment-device channel through-hole 20d, and the air/water feed through-hole 45 formed in the distal-end constituent member 20, the image-capturing unit 32, the bundle of light-guide fibers 42, the treatment-device channel tube 49, and the water feed port metal 46 are disposed, respectively. In addition, the distal-end bending-portion joint piece 51a is arranged on the distal-end bending-portion joint piece arrangement stage 71 incorporated in the distal-end constituent member 20. In this situation, the distal-end bending-portion joint piece 51a is disposed in such a way that the inner surface thereof is adjacent to the image-capturing unit 32.

Provided the notched portion 75b is not formed, it is required to enlarge the inner diameter of the distal-end bending-portion joint piece 51a, by a dimension corresponding to the wall thickness of the peripheral wall, of the distal-end bending-portion joint piece arrangement stage 71, that is not cut off. In other words, that inner diameter is rendered larger than that of the distal-end bending-portion joint piece 51a illustrated in FIG. 11.

In addition, by forming the notched portions 75b, 75c, 75d, and 75e, a first protrusion portion 71a and a second protrusion portion 71b are provided, as illustrated in FIGS. 4 and 10, in the proximal portion of the distal-end bending-portion joint piece arrangement stage 71. The portions, in FIG. 10, indicated by the crosshatch represent the end profiles of the protrusion portions 71a and 71b incorporated in the distal-end bending-portion joint piece arrangement stage 71. In addition, the notched portions 75b, 75c, 75d, and 75e illustrated in FIGS. 10 and 11 are different in depth; the notched portions 75b and 75d are formed up to the position in the vicinity of the distal-end arrangement position of the distal-end bending-portion joint piece 51a. Additionally, the image-capturing unit 32 is held by the first and second protrusion portions 71a and 71b in such a way that the portions thereof that are oriented to bending directions in which the image-capturing unit 32 is liable to be damaged are protected.

As described above, in forming the distal-end bending-portion joint piece arrangement stage in the distal-end constituent member, the center position OA for defining the contour of the distal-end formation portion corresponding to the contour of the distal-end constituent member and the center position OB for defining the peripheral face of the distal-end bending-portion joint piece arrangement stage on which the distal-end bending-portion joint piece is arranged are made eccentric with each other. By making the distal-end bending-portion joint piece small-diameter to eliminate the state that the distal-end bending-portion joint piece arranged on the distal-end bending-portion joint piece arrangement stage and the distal-end formation portion are offset from each other, the diameter of the bending portion including the distal-end bending-portion joint piece and joint pieces that are consecutively connected with the distal-end bending-portion joint piece can be reduced.

As a result, the fact is eliminated that, in the case where the distal end portion and the bending portion are consecutively arranged in such a way that the center axis of the bending portion offset from the center axis of the distal end portion, the outline of the bending portion evenly becomes larger than the peripheral face of the distal end portion. In other words, outline variation portions are eliminated that are evenly formed around the overall circumference of the insertion portion. Accordingly, by appropriately implementing twisting manipulation and bending manipulation, the operator can more smoothly insert the insertion portion into body cavities.

Moreover, by cutting out the peripheral walls, of the distal-end bending-portion joint piece arrangement stage, that are the outer circumference portions of the through-holes into which inner devices are inserted, and thereby making the distal-end bending-portion joint piece closer to the inner devices, the eccentricity of the center position OB with respect to the center position OA is made further larger and the diameter of the distal-end bending-portion joint piece is further reduced, whereby the diameter of the bending portion can be reduced. Accordingly, the insertion portion can more smoothly be inserted into body cavities.

In addition, it is to be understood that the present invention is not limited to the foregoing embodiment, and modifications to the foregoing embodiments may be implemented without departing from the spirit and scope of the present invention.

What is claimed is:

1. An endoscope in which a hard distal end portion and a bending portion are arranged consecutively with each other at the distal side of an insertion portion, wherein, when being coupled, the center axes of the distal end portion and the bending portion are eccentric with each other.

2. An endoscope provided with a hard distal end portion and a bending portion that is arranged consecutively with a proximal side of the distal end portion and formed of a plurality of bending-portion joint pieces connected in series, the endoscope comprising:
a distal-end bending-portion joint piece incorporated in the foremost end of the bending portion; and
a distal-end bending-portion joint piece arrangement stage that is provided at a proximal side of the distal-end constituent member incorporated in the distal end portion and on which the distal-end bending-portion joint piece is integrally provided, wherein the center position of the distal-end bending-portion joint piece arrangement stage formed in the distal-end constituent member and a center position that is a reference position for defining the outline of the distal-end constituent member are eccentric with each other.

3. The endoscope according to claim 2, wherein the distal-end constituent member includes an observation-optical-system through-hole for arranging therein at least a image-capturing device and a wall portion is included at a position that is between the circumference of the observation-optical-system through-hole and the distal-end bending-portion joint piece arrangement stage and that is oriented to the bending direction of the bending portion.

4. An endoscope in which an air/water feed nozzle is disposed in a nozzle-arrangement hole formed in a distal-end constituent member, the endoscope comprising:
a nozzle fixation portion disposed fixedly into the nozzle-arrangement hole, by means of a fixation member, the nozzle fixation portion including a cylindrical engagement portion having a circular cross section and a rotation prevention portion formed in such a way as to be situated at the side of the air/water feed nozzle, to have a diameter larger than that of the cylindrical engagement portion, and to have a flat portion; and
a nozzle portion that is situated at the distal-end side of the nozzle fixation portion and protrudes in a direction perpendicular to the longitudinal axis of the air/water feed nozzle, the nozzle portion including an abutting surface that abuts on the distal endface of the distal-end constituent member and a groove portion that is formed by cutting off the middle part of the abutting surface and forms an emission outlet.

5. An endoscope in which an observation optical system and an air/water feed nozzle are arranged at a distal endface of the distal-end constituent member, wherein the distal-end constituent member includes at the distal endface thereof a first plane in which the observation optical system is disposed and a second plane on which the abutting surface of the air/water feed nozzle abuts and the first plane is ahead with respect to the second plane, in the axis direction.

6. The endoscope according to claim 5, wherein an observation-window surface of the observation optical system is situated behind the distal-end surface of an emission outlet of the air/water feed nozzle, in the axis direction.

7. The endoscope according to claim 4, wherein the endoscope is configured in such a way that, at the distal endface of the distal-end constituent member, a first plane in which the observation optical system is disposed and a second plane on which the abutting surface of the air/water feed nozzle abuts are formed, and the first plane is ahead with respect to the second plane, in the axis direction.

8. The endoscope according to claim 4, wherein the flat portion of the nozzle fixation portion also plays the role of a positioning portion for making the emission outlet of the nozzle portion face the surface of the observation optical system.

9. The endoscope according to claim 7, wherein the endoscope is configured in such a way that, at the distal endface of the distal-end constituent member, the first plane in which the surface of the observation optical system is disposed and the second plane in which the nozzle portion of the air/water feed nozzle is disposed are provided, and, in consideration of the viewing angle of the observation optical system, the level difference between the first and second planes is set in such a way that part of the air/water feed nozzle is prevented from being contained in the viewing angle.

10. The endoscope according to claim 9, wherein the endoscope is configured in such a way that, by making the abutting surface abut on the second plane of the distal-end constituent member, the air/water feed nozzle is disposed fixedly, and the distance between the abutting surface and the bottom surface of the groove portion, that is a depth of the groove portion provided in the water feed nozzle is set in such a way that the groove-portion bottom is situated ahead with respect to the surface of the observation optical system.
